(19) United States
(12) Patent Application Publication
(10) Pub. No.: US 2007/0155770 A1
(43) Pub. Date: Jul. 5, 2007

(54) Treatment of anoragasmia or delayed orgasm

(75) Inventors: Shlomo Melmed, Los Angeles, CA (US); Waguih IsHak, Beverly Hills, CA (US); Daniel Berman, Los Angeles, CA (US)

Correspondence Address: DAVIS WRIGHT TREMAINE LLP, 865 FIGUEROA STREET, SUITE 2400, LOS ANGELES, CA, 90017-2566, US

(73) Assignee: Cedars-Sinai Medical Center, Los Angeles, CA (US)

(21) Appl. No.: 11/324,983

(22) Filed: Jan. 3, 2006

(51) Int. Cl. A61K 31/48

(52) U.S. Cl. 514/288

(57) ABSTRACT

Described herein are compositions and methods for use in the treatment of anorgasmia and/or delayed orgasm. In various embodiments, the compositions include a dopamine receptor agonist and/or an antiprolactin. Cabergoline is a dopamine receptor agonist suitable for use in connection with particular embodiments of the invention. Its analogs, salts, equivalents, and derivatives may be useful as well. Also described are kits including a dopamine receptor agonist and/or an antiprolactin for use in connection with the aforementioned therapeutic methods.

### FIELD OF INVENTION

This invention relates to methods using dopamine receptor agonists and/or antiprolactin compositions for the treatment of anorgasmia or delayed orgasm.

### BACKGROUND OF THE INVENTION

Epidemiological studies show that 25% of women and 10% of men suffer from anorgasmia or delayed orgasm. In particular, female sexual dysfunction is considered to be as high as 43% in the U.S. population based on a study published in the Journal of the American Medical Association (JAMA, Vol. 281, No. 6 (Feb. 10, 1999)), with anorgasmia being the second most common sexual disorder.

Current treatments for anorgasmia or delayed orgasm consist of couples counseling, couples therapy and self-help books. Additional therapies include stimulation devices. Psychotherapy as a treatment option for anorgasmia or delayed orgasm is largely dependent on the motivation of the individual and the couple, and has not been effective in a significant portion of patients with anorgasmia. Furthermore, psychosocial interventions consume a lot of time and effort. Currently, there are no medications for treatment for anorgasmia or delayed orgasm. Thus, there exists a need for methods to biologically treat anorgasmia and delayed orgasm.

### SUMMARY OF INVENTION

The following embodiments and aspects thereof are described and illustrated in conjunction with methods and are meant to be exemplary and illustrative, not limiting in scope. In various embodiments, one or more of the above-described problems have been reduced or eliminated, while other embodiments are directed to other improvements.

In accordance with various embodiments, compositions including dopamine receptor agonists and/or antiprolactin compositions may be used to treat anorgasmia or delayed orgasm in a mammal. In one embodiment, the mammal is human. In another embodiment the dopamine receptor agonist is a long acting dopamine receptor agonist. In a particular embodiment the long acting dopamine receptor agonist is cabergoline.

Further embodiments include methods using dopamine receptor agonists and/or antiprolactin compositions to treat anorgasmia or delayed orgasm in a mammal such as a human. In one embodiment the dopamine receptor agonist is a long acting dopamine receptor agonist. In various embodiments, the human may be hyperprolactinemic. In other embodiments, the prolactin level in the human is normal. In a particular embodiment the long acting dopamine receptor agonist is cabergoline.

The present invention is also directed to a kit for the treatment of anorgasmia or delayed orgasm. The exact nature of the components configured in the inventive kit depends on its intended purpose. For example, some embodiments are configured for the purpose of treating anorgasmia or delayed orgasm. Instructions for use may be included in the kit. “Instructions for use” typically include a tangible expression describing the technique to be employed in using the components of the kit to effect a desired outcome, such as to enable the subject to reach the orgasm phase of a sexual response cycle. Optionally, the kit also contains other useful components.

Other features and advantages of the invention will become apparent from the following detailed description.

### DESCRIPTION OF INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); and March, Advanced Organic Chemistry Reactions, Mechanisms and Structure 4th ed., J. Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Anorgasmia” as used herein refers to the failure to reach or achieve the orgasm (climax) phase of a sexual response cycle.

“Delayed orgasm” as used herein refers to a delay or difficulty for the subject to reach the orgasm phase of a sexual response cycle.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and minors, whether male or female, are intended to be including within the scope of this term.

“Sexual activity” as used herein refers to activity that places a subject in a stage of the sexual response cycle. Examples include but are not limited to, vaginal or anal intercourse, masturbation, oral sex, cunnilingus, fellatio and other non-coital activities.

“Sexual response cycle” as used herein includes but is not limited to the following phases: excitement, plateau, orgasm, and resolution.

“Therapeutically effective amount” as used herein, with respect to a patient with anorgasmia, refers to that amount which is capable of enabling the patient to reach the orgasm phase of a sexual response cycle. With respect to a patient with delayed orgasm, a “therapeutically effective amount” as used herein refers to that amount which is capable of reducing the delay or difficulty for the subject to reach the orgasm phase of a sexual response cycle. A therapeutically effective amount can be determined on an individual basis and will be based, at least in part, on consideration of the physiological characteristics of the mammal, the type of delivery system or therapeutic technique used and the time of administration relative to treatment for anorgasmia or delayed orgasm.

“Treatment” and “treating,” as used herein refer to both therapeutic treatment and prophylactic or preventative measures, wherein the object is to decrease or end the incidences of anorgasmia or delayed orgasm, even if the treatment is ultimately unsuccessful.

The invention relates to compositions and methods useful in the medical arts. The composition of the present invention includes dopamine receptor agonists and/or antiprolactin compositions as an orgasm facilitating agent. In one embodiment, long acting dopamine receptor agonists may be used. Cabergoline is one such agent; chemical analogs, pharmaceutically acceptable salts, derivatives, equivalents, and the like may also be used. Oral cabergoline is available under the trade name, Dostinexe from Pfizer, Inc. (New York, N.Y.). Cabergoline has been used to treat medical problems such as hyperprolactinemic disorders, either idiopathic or due to pituitary adenomas. Cabergoline has also been described as useful in the treatment of Parkinsonism and Restless Leg Syndrome. The inventors have discovered that cabergoline may be used for a new therapeutic purpose: for the treatment of anorgasmia or delayed orgasm.

The hormone, prolactin, is secreted at high levels only after orgasm in both men and women. High prolactin levels prevent further orgasms. While not wishing to be bound by any particular theory, the inventors believe that cabergoline works as an anti-prolactin medication helping to facilitate orgasm.

The inventors have used cabergoline for the treatment of anorgasmia or delayed orgasm in patients with hyperprolactinemia and in patients with normal levels of prolactin, as described in the ensuing Examples.

In accordance with various embodiments, compositions, including dopamine receptor agonists and/or antiprolactin compositions, may be used to treat anorgasmia or delayed orgasm in a mammal. In one embodiment, the mammal is human. In another embodiment the dopamine receptor agonist is a long acting dopamine receptor agonist. In a particular embodiment the long acting dopamine receptor agonist is cabergoline, or an analog, pharmaceutically acceptable salt, derivative, or equivalent of the same.

Further embodiments include methods using the compositions to treat anorgasmia or delayed orgasm in a mammal such as a human. In various embodiments, the human may have hyperprolactinemia. In other embodiments, the prolactin level in the human may be normal. In various embodiments, the cabergoline is administered to the human about 3 to about 4 hours prior to sexual activity. In further embodiments, the human is administered at least about 0.25 mg, and, in some embodiments, from about 0.5 mg to about 1.0 mg of cabergoline or an analog, pharmaceutically acceptable salt, derivative, or equivalent of the same on each dosing occasion.

The compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000). In one particular embodiment, the therapeutically effective amount of the composition may be about 0.5 mg to about 1.0 mg administered about 3 to about 4 hours prior to sexual activity where it may be advantageous to treat anorgasmia or delayed orgasm (e.g., intercourse). In one particular aspect the composition comprises about 0.5 mg to about 1.0 mg of cabergoline, or an analog, pharmaceutically acceptable salt, derivative, or equivalent of the same.

In various embodiments of the present invention, the inventive compositions and/or the overall treatment regimen (even if not embodied in a single composition) may include additional active ingredients to further treat anorgasmia or delayed orgasm and/or to mitigate the side effects associated with the dopamine receptor agonists and/or antiprolactins. Such additional active ingredients will be readily recognized by those of skill in the art.

In various embodiments, the present invention provides pharmaceutical compositions including a pharmaceutically acceptable excipient along with a therapeutically effective amount of cabergoline. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The present invention is also directed to a kit for the treatment of anorgasmia or delayed orgasm. The kit is useful for practicing the inventive method of treating anorgasmia or delayed orgasm. The kit is an assemblage of materials or components, including at least one of the inventive compositions. Thus, in some embodiments the kit contains a composition including cabergoline, or an analog, pharmaceutically acceptable salt, derivative, or equivalent of the same, as described above.

The exact nature of the components configured in the inventive kit depends on its intended purpose. For example, some embodiments are configured for the purpose of treating anorgasmia or delayed orgasm. In one embodiment, the kit is configured particularly for the purpose of treating mammalian subjects. In another embodiment, the kit is configured particularly for the purpose of treating human subjects.

Instructions for use may be included in the kit. “Instructions for use” typically include a tangible expression describing the technique to be employed in using the components of the kit to effect a desired outcome, such as to enable the subject to experience an orgasm. Optionally, the kit also contains other useful components, such as, diluents, buffers, pharmaceutically acceptable carriers, syringes, catheters, applicators, pipetting or measuring tools, contraceptives or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in medical treatment kits. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing cabergoline, or an analog, pharmaceutically acceptable salt, derivative, or equivalent of the same. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

An 80 year-old male patient, with normal levels of prolactin, presented with a diagnosis of anorgasmia and had not been able to have an orgasm for 1.5 years. Biopsychosocial evaluation for sexual dysfunction was performed on this patient. The patient was instructed to ingest 0.5 mg of Dostinex® (cabergoline) 3-4 hours prior to sexual intercourse. The patient reported that orgasmic ability was resumed and that he was able to have an orgasm twice per week.

### Example 2

A 53 year-old female patient having been diagnosed with anorgasmia and hyperprolactinemia for approximately 8 years presented for treatment. She was instructed to ingest 0.5 mg of Dostinex® (cabergoline) 3-4 hours prior to sexual intercourse. The patient reported superior results leading to restoration of orgasm and overall sexual satisfaction.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method for treating anorgasmia or delayed orgasm in a mammal, comprising:
providing a composition comprising a dopamine receptor agonist; and
administering a therapeutically effective amount of the composition to the mammal.

2. The method of claim 1, wherein the mammal is a human.

3. The method of claim 1, wherein the dopamine receptor agonist is selected from the group consisting of cabergoline, an analog of cabergoline, a pharmaceutically acceptable salt of cabergoline, an equivalent of cabergoline, a derivative of cabergoline, and combinations thereof.

4. The method of claim 1, wherein the administering a therapeutically effective amount of the composition further comprises:
administering a therapeutically effective amount of the composition prior to sexual activity by the mammal.

5. The method of claim 1, wherein the administering a therapeutically effective amount of the composition further comprises:
administering a therapeutically effective amount of the composition about 3 to about 4 hours prior to sexual activity by the mammal.

6. The method of claim 1, wherein the therapeutically effective amount is about 0.5 mg to about 1.0 mg.

7. The method of claim 1, wherein administering a therapeutically effective amount of the composition further comprises:
delivering the composition via an oral route of administration.

8. A method for treating anorgasmia or delayed orgasm in a human, comprising:
providing an amount of cabergoline; and
administering a therapeutically effective amount of the cabergoline to a human about 3 to about 4 hours prior to sexual activity by the human.

9. The method of claim 8, wherein the therapeutically effective amount of cabergoline is about 0.5 mg to about 1.0 mg.

10. A method for treating anorgasmia or delayed orgasm in a mammal, comprising:
providing a composition comprising an antiprolactin; and
administering a therapeutically effective amount of the composition to the mammal.

11. The method of claim 10, wherein the administering a therapeutically effective amount of the composition further comprises:
administering a therapeutically effective amount of the composition prior to sexual activity by the mammal.

12. The method of claim 10, wherein the administering a therapeutically effective amount of the composition further comprises:
administering a therapeutically effective amount of the composition about 3 to about 4 hours prior to sexual activity by the mammal.

13. The method of claim 10, wherein the therapeutically effective amount is about 0.5 mg to about 1.0 mg.

14. A kit, comprising:
a composition, comprising a dopamine receptor agonist and/or an antiprolactin; and
instructions for the use of the composition to treat anorgasmia or delayed orgasm in a human.

15. The kit of claim 14, wherein the dopamine receptor agonist is selected from the group consisting of cabergoline, an analog of cabergoline, a pharmaceutically acceptable salt of cabergoline, an equivalent of cabergoline, a derivative of cabergoline, and combinations thereof.

16. The kit of claim 14, wherein the instructions for the use of the composition to treat anorgasmia or delayed orgasm comprise:
instructions to administer a therapeutically effective amount of the dopamine receptor agonist and/or antiprolactin.

17. The kit of claim 16, wherein the therapeutically effective amount is about 0.5 mg to about 1.0 mg.

18. The kit of claim 14, wherein the instructions for the use of the composition to treat anorgasmia or delayed orgasm comprise:
instructions to administer the dopamine receptor agonist and/or antiprolactin about 3 to about 4 hours prior to sexual activity by the human.
